(19) United States
(12) Patent Application Publication
(10) Pub. No.: US 2022/0280684 A1
(43) Pub. Date: Sep. 8, 2022

(54) BIOCOMPATIBLE HYDROGEL COMPRISING HYALURONIC ACID AND POLYETHYLENE GLYCOL

(71) Applicant: Kyungpook National University Industry-Academic Cooperation Foundation, Daegu (KR)

(72) Inventors: Jeong Soo Yoo, Daegu (KR); Woong Hee Lee, Daegu (KR); Wanwook Kim, Daegu (KR)

(21) Appl. No.: 17/629,607

(22) PCT Filed: Jul. 24, 2020
(86) PCT No.: PCT/KR2020/009788

§ 371 Date: Jan. 24, 2022

(30) Foreign Application Priority Data
Jul. 24, 2019 (KR) 10-2019-0089858

(51) Int. Cl. A61L 26/00; A61L 27/26; A61L 27/52; C08L 71/02; C08L 5/08; C08J 3/075; C08J 3/24; C08J 3/28

(52) U.S. Cl. CPC A61L 26/008; A61L 26/0052; A61L 26/0076; A61L 27/26; A61L 27/52; C08L 71/02; C08L 5/08; C08J 3/075; C08J 3/246; C08J 3/28; A61L 2400/06; A61L 2430/34; C08L 2203/02; C08L 2312/00; C08J 2305/08; C08J 2371/02; C08J 2405/08; C08J 2471/02

(57) ABSTRACT

The present invention relates to a biocompatible hydrogel comprising hyaluronic acid and polyethylene glycol, and more particularly, to a biocompatible hydrogel prepared by inducing inter-molecular and/or intra-molecular cross-linking of hyaluronic acid and polyethylene glycol only by irradiating radiation without adding a reactor, a chemical cross-linking agent, or the like, a method for preparing the same, and the use thereof.

### TECHNICAL FIELD

This application claims the priority of Korean Patent Application No. 10-2019-0089858, filed on Jul. 24, 2019, the entirety of which is a reference of the present application.

The present invention relates to a biocompatible hydrogel comprising hyaluronic acid and polyethylene glycol, and more particularly, to a biocompatible hydrogel prepared by inducing inter-molecular and/or intra-molecular cross-linking of hyaluronic acid and polyethylene glycol merely by irradiating radiation without adding a reactor, a chemical cross-linking agent, or the like, a method for preparing the same, and use thereof.

### BACKGROUND ART

Recently, injectable hydrogels have received much attention in a medical field, and are expected to be widely used, such as a release system of a physiologically active material from a medical filler, and organ/tissue regeneration using a three-dimensional structure. These injectable hydrogels have an advantage of being simply injected into the body using a syringe or the like without a surgical procedure. In general, the injectable hydrogels has the same characteristics as a fluid outside the body to be implanted using a syringe, and needs to have fluidity for the convenience of operation, and is injected into the body, and should be gelled in one place so that the shape thereof is not in disorder through chemical or physical cross-linking. That is, after implantation, the injectable hydrogels serves as a drug delivery system for continuous release of cells or drugs and a support for maintaining cell growth, or needs to exhibit a cosmetic effect by maintaining a certain shape in the skin soft tissue.

On the other hand, such a hydrogel has been generally prepared by adding and cross-linking a chemical material such as a cross-linking agent and/or a curing agent to a polymer material. However, since the cross-linking agent and/or the curing agent used in the cross-linking reaction is harmful to the living body, when hydrogels prepared using such a cross-linking agent and/or curing agent are used in the living body, there is a problem that the hydrogels may cause a harmful action. In particular, such hydrogels are unsuitable to be used as medical and pharmaceutical materials, such as wound dressings, drug delivery carriers, contact lenses, cartilages, intestinal anti-adhesion agents, and the like. In addition, when the cross-linking agent and/or the curing agent is used, the remaining cross-linking agent and/or curing agent in the hydrogel needs to be removed after preparing the hydrogel, so that the preparing process is complicated and the preparing cost is increased.

Accordingly, efforts to prepare a polymer-derived hydrogel without using the cross-linking agent and/or curing agent are continuing, and as a result of these efforts, a result of preparing the hydrogel by irradiating radiation to a synthetic polymer has been reported.

However, since the synthetic polymer-derived hydrogel is not suitable to be used for medical purposes in terms of biocompatibility and biodegradability, it is required to develop a hydrogel formed solely by intra-molecular or inter-molecular cross-linking of biocompatible molecules without using a cross-linking agent, a curing agent, an organic solvent, etc.

On the other hand, hyaluronic acid is a biopolymer material as a kind of polysaccharides in which repeating units consisting of N-acetyl-glucosamine and D-glucuronic acid are linearly connected. The hyaluronic acid is first isolated from the fluid that fills the eyeballs of animals and then is known to exist abundantly in the placenta of animals, a synovial fluid of joints, a pleural fluid, skin, and a comb of the rooster, and produced even in Streptococcus equi, Streptococcus zooepidemecus, and the like of Streptococcus microorganisms.

The hyaluronic acid has been widely used not only for cosmetic applications such as cosmetic additives, but also for various pharmaceutical uses such as ophthalmic surgical aids, joint function improving agents, drug delivery materials, and eye drops due to excellent biocompatibility and high viscoelasticity in a solution. However, since hyaluronic acid itself is easily decomposed in vivo or under conditions such as acid and alkali and its use is very limited, in the preparation of a hyaluronic acid-based hydrogel, it is general to add a chemical cross-linking agent (WO2013/055832).

Particularly, it is well known in the art that biocompatible polymers such as carboxymethyl cellulose, methyl cellulose, hydroxyethyl cellulose, and carboxymethyl starch may form a gel by irradiating radiation (Nuclear Instruments and Methods in Physics Research B 208 (2003) 320-324, Carbohydrate Polymers 112 (2014) 412-415, Nuclear Instruments and Methods in Physics Research B 211 (2003) 533-544, etc.). In the case of hyaluronic acid, the molecular weight is reduced and the viscosity is reduced by irradiating the radiation, so that the degradation reaction occurs easily (Korea Patent Publication No. 10-2008-0086016, etc.), and a hyaluronic acid-based hydrogel prepared through radiation irradiation, that is, a hyaluronic acid-based hydrogel prepared only by irradiation without adding a chemical cross-linking agent, an organic chemical, etc. is not yet provided.

### DISCLOSURE

### Technical Problem

Therefore, the present inventors repeated many studies to provide a hyaluronic acid-based biocompatible hydrogel prepared only by irradiating radiation without using a chemical cross-linking agent, an organic chemical material, and the like, and as a result, found that another biocompatible polymer, polyethylene glycol was used together to prepare a hyaluronic acid-polyethylene glycol hydrogel exhibiting various physical properties under a specific preparation condition, and then completed the present invention.

Accordingly, an object of the present invention is to provide a hydrogel formed solely by inter-molecular cross-linking, intra-molecular cross-linking, or inter-molecular and intra-molecular cross-linking of hyaluronic acid and polyethylene glycol (PEG).

Another object of the present invention is to provide a method of preparing a hydrogel formed solely by inter-molecular cross-linking, intra-molecular cross-linking, or inter-molecular and intra-molecular cross-linking of hyaluronic acid and polyethylene glycol (PEG) including: (a) preparing a solution by adding hyaluronic acid and polyethylene glycol to water; and (b) inducing the cross-linking of said materials by irradiating radiation to the solution prepared in step (a).

Yet another object of the present invention is to provide a cell carrier, a drug carrier, an anti-adhesion agent, a cell support, a dental filler, an orthopedic filler, a wound dressing or a skin filler including the hydrogel.

Another object of the present invention is to provide a composition for skin application of a wound site comprising the hydrogel as an active ingredient.

In addition, another object of the present invention is to provide a composition for skin application of the wound site consisting of the hydrogel.

In addition, another object of the present invention is to provide a composition for skin application of the wound site essentially consisting of the hydrogel.

Another object of the present invention is to provide use of the hydrogel for preparing an agent for skin application of a wound site.

Another object of the present invention is to provide a method for treating a wound site by applying an effective amount of a composition comprising the hydrogel as an active ingredient onto the skin of a subject in need thereof.

### Technical Solution

In order to achieve the object of the present invention, the present invention provides a hydrogel formed solely by inter-molecular cross-linking, intra-molecular cross-linking, or inter-molecular and intra-molecular cross-linking of hyaluronic acid and polyethylene glycol (PEG).

In order to achieve another object of the present invention, the present invention provides a method of preparing a hydrogel formed solely by inter-molecular cross-linking, intra-molecular cross-linking, or inter-molecular and intra-molecular cross-linking of hyaluronic acid and polyethylene glycol (PEG) including:(a) preparing a solution by adding hyaluronic acid and polyethylene glycol to water; and (b) inducing the cross-linking of said materials by irradiating radiation to the solution prepared in step (a).

In order to achieve another object of the present invention, the present invention provides a cell carrier, a drug carrier, an anti-adhesion agent, a cell support, a dental filler, an orthopedic filler, a wound dressing or a skin filler including the hydrogel.

In order to achieve another object of the present invention, the present invention provides a composition for skin application of a wound site comprising the hydrogel as an active ingredient.

Further, the present invention provides a composition for skin application of the wound site consisting of the hydrogel.

Further, the present invention provides a composition for skin application of the wound site essentially consisting of the hydrogel.

In order to achieve another object of the present invention, the present invention provides use of the hydrogel for preparing an agent for skin application of a wound site.

In order to achieve another object of the present invention, the present invention provides a method for treating a wound site by applying an effective amount of a composition comprising the hydrogel as an active ingredient onto the skin of a subject in need thereof.

Hereinafter, the present invention will be described in detail.

The present invention provides a hydrogel formed solely by inter-molecular cross-linking, intra-molecular cross-linking, or inter-molecular and intra-molecular cross-linking of hyaluronic acid and polyethylene glycol (PEG).

In a method of preparing a hydrogel using a polymer, a cross-linking agent is generally used to induce cross-linking of the polymer. In the case of a method of inducing the cross-linking of polymers using a cross-linking agent, the cross-linking agent may be mixed into the hydrogel because the cross-linking agent mediates inter-molecular or intra-molecular bonding, and there may be a problem in that since the concentration of the cross-linking agent is high, the cross-linking agent may remain in a reactant in an active state, or an unreacted product remaining after the reaction exists, and as a result, a purification process during the hydrogel preparing process is required. In addition, the cross-linking agent remaining in the hydrogel may cause various side effects after administrated into the body. However, the present inventors confirmed that an electron beam is irradiated to a mixture of hyaluronic acid and polyethylene glycol under a specific condition to induce inter-molecular or intra-molecular cross-linking of hyaluronic acid and/or polyethylene glycol and form a hydrogel. A hydrogel formed by only binding of hyaluronic acid and/or polyethylene glycol itself without containing an external material such as a cross-linking agent or metal cations additionally added for physical cross-linking in the molecule has not yet reported in the related art, but is first published by the present inventors through the present invention.

On the other hand, all medical materials as well as polymer materials absolutely require biocompatibility, and this biocompatibility may be distinguished into two aspects. Biocompatibility in a wide sense refers to both a desired function and safety to the living body, and biocompatibility in a narrow sense refers to biosafety to the living body, that is, non-toxicity and sterilization.

However, since the biocompatible hydrogel of the present invention is formed only by inter-molecular or intra-molecular cross-linking of hyaluronic acid and/or polyethylene glycol, there is an advantage that a hyaluronic acid-based hydrogel prepared according to a conventional method has no problems and has very excellent biocompatibility. In addition, since the hydrogel can be prepared by irradiating an electron beam in an aqueous solution without using all organic solvents in the process of preparing the hydrogel of the present invention, contamination or complicated processes that may occur in the preparing process are not required, so that the hydrogel is very useful for industrial use.

That is, the hydrogel provided in the present invention does not bind to any functional group additionally introduced to hyaluronic acid and polyethylene glycol, and no cross-linking agent directly participates in or mediates cross-linking other than hyaluronic acid and polyethylene glycol.

In the present invention, hyaluronic acid, which is a raw material of the biocompatible hydrogel, is very useful as a carrier for drugs, etc. due to a multifunctional group present in its chemical structure, and has more excellent applicability than a synthetic polymer in a medical field due to physicochemical characteristics such as biocompatibility and biodegradability (Materials Science and Engineering C 68 (2016) 964-981).

In the present invention, the hyaluronic acid includes all of hyaluronic acid, a hyaluronic acid salt, or a mixture of hyaluronic acid and a hyaluronic acid salt. The hyaluronic acid salt may be at least one selected from the group consisting of sodium hyaluronic acid, potassium hyaluronic acid, calcium hyaluronic acid, magnesium hyaluronic acid, zinc hyaluronic acid, cobalt hyaluronic acid, and tetrabutylammonium hyaluronic acid, but is not limited thereto.

In the present invention, the polyethylene glycol has many advantages in a drug delivery field and tissue engineering, and representatively, has high solubility in an organic solvent and non-toxicity and exhibits excellent biocompatibility without rejection to an immune action, and may easily capture and release drugs as a drug carrier, and has been used in the pharmaceutical formulation industry as a material approved for use by the US Ministry of Food and Drug Safety for use in the human body. In addition, the polyethylene glycol improves the biocompatibility of polymers used for blood contact among hydrophilic polymers and has the greatest effect of inhibiting protein adsorption to have many applications as a biomaterial [J. H. Lee, J. Kopecek, and J. D. Andrade, J. Biomed. Mater. Res., 23 (1989) 351].

The hydrogel provided by the present invention may be particularly characterized to be prepared by a method comprising the steps of:

(a) preparing a solution by adding hyaluronic acid and polyethylene glycol to water; and

(b) inducing the cross-linking of said materials by irradiating radiation to the solution prepared in step (a).

According to various embodiments, the present inventors have established a preparation condition of a hydrogel consisting only of inter-molecular cross-linking and/or intra-molecular cross-linking of hyaluronic acid and polyethylene glycol through irradiation with radiation.

According to an embodiment of the present invention, it was confirmed that it is very important to combine various conditions to generate a hydrogel by inducing inter-molecular cross-linking and/or intra-molecular cross-linking of hyaluronic acid and polyethylene glycol using irradiation with radiation. Specifically, it was confirmed that when the molecular weight/concentration of hyaluronic acid, the molecular weight/concentration of polyethylene glycol, the energy irradiation amount and the energy intensity do not satisfy certain conditions, the hydrogel is not formed at all. In addition, it was confirmed that the preparation of hydrogels exhibiting various physical properties is possible through appropriate control of these conditions.

According to another embodiment of the present invention, when polyethylene glycol having a molecular weight of 2 to 50 kDa is added to water at a concentration of 0.6 to 3% (w/v), hydrogels with various physical properties are formed through the control of the radiation amount and intensity regardless of the concentration and molecular weight of hyaluronic acid to be added together.

In particular, it was confirmed that as the molecular weight of polyethylene glycol used to prepare the hydrogel increased, there was the tendency to generate the hydrogel even at a lower radiation dose condition. In addition, it was confirmed that as the intensity of the irradiated radiation energy is increased, the hydrogel was formed even under a polyethylene glycol aqueous solution at a lower concentration.

Therefore, in step (a) of the present invention, polyethylene glycol having a molecular weight of 2 to 50 kDa may be used, preferably polyethylene glycol having a molecular weight of 3 to 40 kDa may be used, and most preferably, polyethylene glycol having a molecular weight of 3 to 35 kDa may be used.

In addition, in step (a) of the present invention, polyethylene glycol may be added to water at a concentration of 0.6 to 3% (w/v), preferably at a concentration of 0.8 to 2% (w/v), more preferably at a concentration of 0.8 to 1.5% (w/v), and most preferably at a concentration of 0.9 to 1.2% (w/v).

Accordingly, the molecular weight of hyaluronic acid used in step (a) of the present invention and the concentration of hyaluronic acid in the aqueous solution are not particularly limited, but hyaluronic acid having a molecular weight of 50 to 3000 kDa may be used, preferably, hyaluronic acid having a molecular weight of 70 to 2700 kDa may be used, and most preferably, hyaluronic acid having a molecular weight of 100 to 2500 kDa may be used.

In a preferred embodiment of the present invention, the concentration (w/v) of polyethylene glycol in step (a) is the same as or greater than the concentration (w/v) of hyaluronic acid.

Meanwhile, the step (b) of the present invention is a step of inducing the cross-linking of said materials by irradiating radiation to the solution prepared in step (a).

The hydrogel formed by the radiation irradiation has an advantage that there is no problem of residual toxicity present in the hydrogel prepared by a chemical method, and a sterilization effect may be obtained at the same time as cross-linking. In this case, the radiation used may be at least one selected from the group consisting of gamma rays, ultraviolet rays, X-rays and electron beams, and preferably electron beams.

According to an embodiment of the present invention, it was confirmed that the dose and/or energy intensity of the radiation irradiated to form the hydrogel in step (b) may vary depending on the molecular weight/concentration of hyaluronic acid and the molecular weight/concentration of polyethylene glycol used in step (a). In addition, it was confirmed that the physical properties of the hydrogel vary depending on the dose and/or energy intensity of the irradiated radiation even under the conditions in which the hydrogel is formed. It was confirmed that a more rigid hydrogel was formed as the radiation dose increased within a predetermined range, but it was confirmed that when the radiation dose exceeded the predetermined range, the cross-linking in the hydrogel was partially cleaved so that the hydrogel with a reduced degree of rigidity was formed.

Although the range of the radiation dose and energy intensity irradiated in step (b) of the present invention is not particularly limited, the radiation dose may be preferably 2 to 500 kGy, more preferably 5 to 300 kGy, most preferably 5 to 200 kGy. In addition, the energy intensity of the radiation may be 0.5 to 20 MeV, preferably 1 to 10 MeV, much more preferably 1 to 5 MeV, and most preferably 1 to 2.5 MeV.

Specific preparation conditions for preparing the hydrogel provided in the present invention, that is, practical examples of combining the molecular weight/concentration of hyaluronic acid, the molecular weight/concentration of polyethylene glycol, the radiation dose, and the radiation energy intensity are specifically presented in Examples of the present invention.

Further, the present invention provides a method of preparing a hydrogel formed solely by inter-molecular cross-linking, intra-molecular cross-linking, or inter-molecular and intra-molecular cross-linking of hyaluronic acid and polyethylene glycol (PEG) including the following steps:

(a) preparing a solution by adding hyaluronic acid and polyethylene glycol to water; and

(b) inducing the cross-linking of said materials by irradiating radiation to the solution prepared in step (a).

A detailed description of each step of the preparation method may be applied in the same manner as described above.

The present invention provides a cell carrier, a drug carrier, an anti-adhesion agent, a cell support, a dental filler, an orthopedic filler, a wound dressing (sheet type, gel type, spray type, cream type, etc.) or a skin filler including the hydrogel.

In the present invention, since it is possible to provide a hydrogel satisfying various physical properties by changing preparation conditions within the above-described range according to a desired use, it is possible to provide a hydrogel suitable for each use with viscoelasticity and an in-vivo decomposition period. In addition, since any chemical cross-linking agent and organic chemical material are not used during the preparing process, the biocompatibility is very excellent, so that the hydrogel may be utilized for various purposes.

Since the biocompatible hydrogel has been used variously for a cell carrier, a drug carrier, an anti-adhesion agent, a cell support, a dental filler, an orthopedic filler, a wound dressing (sheet type, gel type, spray type, cream type, etc.), a skin filler, or the like. Since the research thereon has been actively conducted in the art, it is obvious to those skilled in the art that the hydrogel provided in the present invention may also be utilized for the uses.

According to an embodiment of the present invention, it has been confirmed that the hydrogel provided in the present invention has very excellent properties of maintaining its volume and shape for a predetermined period in the body to have the possibility of application as a skin filler.

Accordingly, the hydrogel of the present invention is preferably injected into the dermis layer in the skin to be useful as a skin filler for improving wrinkles, improving the lip contour, improving acne scars, and filling skin depressions and/or scars.

The cell carrier, the drug carrier, the anti-adhesion agent, the cell support, the dental filler, the orthopedic filler, the wound dressing (sheet type, gel type, spray type, cream type, etc.) or the skin filler provided in the present invention may additionally include various general additives in addition to the hydrogel. Although types of these additives are not particularly limited, for example, dyes, color pigments, vegetable oils, thickeners, pH adjusters, osmotic pressure adjusters, vitamins, antioxidants, inorganic salts, preservatives, solubilizers, isotonic agents, suspending agents, emulsifiers, stabilizers, anesthetics, and the like may be included.

Further, the present invention provides a composition for skin application of a wound site comprising the hydrogel as an active ingredient.

The ‘wound’ of the present invention means a state in which the continuity of the tissue is destroyed by an external pressure. The wound includes abrasions, bruises, lacerations, cuts caused by knives, and the like.

The composition for skin application of the wound site may additionally include known drugs, disinfectants, etc. capable of helping in the healing of wounds, and may be formulated as a wound dressing and used as a sheet type, gel type, spray type or cream type wound dressing.

In one aspect of the present invention, the composition for skin application of the wound site may include the hydrogel of the present invention described above without limitation, but may include a hydrogel prepared using hyaluronic acid with preferably a molecular weight of 500 kDa or more, and most preferably a molecular weight of 1000 kDa or more.

In another aspect of the present invention, the composition for skin application of the wound site may include the hydrogel of the present invention described above without limitation, but may include a hydrogel at a concentration ratio (w/v) of hyaluronic acid and polyethylene glycol of 1:1 to 4, preferably 1:1 to 3, most preferably 1:1 to 2.

The present invention provides use of the hydrogel for preparing an agent for skin application of the wound site.

The present invention provides a method of treating a wound site by applying an effective amount of a composition comprising the hydrogel as an active ingredient onto the skin of a subject in need thereof.

The ‘effective dose’ of the present invention means an amount which exhibits effects of improving, treating, detecting, and diagnosing of the wound, or inhibiting or reducing the progression of the wound when administered to the subject. The ‘subject’ may be animals, preferably, mammals, particularly animals including humans and may also be cells, tissues, and organs derived from animals. The subject may be patients requiring the effects.

The ‘treatment’ of the present invention comprehensively refers to improving a wound site or symptoms caused by the wound, and may include treating or substantially preventing the wound, or improving the conditions thereof and includes alleviating, treating or preventing a symptom or most of symptoms derived from the disease, but is not limited thereto.

The term “comprising” herein is used in the same meaning as “including” or “characterized by”, and does not exclude additional ingredients or steps of the method which are not specifically mentioned in the composition or the method according to the present invention. The term “consisting of” means excluding additional elements, steps or ingredients, etc., unless otherwise described. The term “consisting essentially of” means including materials or steps which do not substantially affect basic properties thereof in addition to the described materials or steps within the range of the composition or the method.

### Advantageous Effects

Since the hydrogel of the present invention is prepared by inducing inter-molecular and/or intra-molecular cross-linking of hyaluronic acid and polyethylene glycol through an electron beam, there is no risk of toxicity in the human body due to the mixing of an organic solvent or a cross-linking agent, a separate purification process is not required during the preparing process, and mass production is possible only by irradiating the electron beam in a short time, so that it is very excellent even in terms of productivity. In addition, since the hydrogel of the present invention has very excellent biocompatibility, it may be very usefully used in the development of cell carriers, drug carriers, anti-adhesion agents, cell supports, dental fillers, orthopedic fillers, wound dressings, skin fillers, or the like.

### DESCRIPTION OF DRAWINGS

FIG. 1 is a diagram visually observing whether or not a hydrogel is formed by concentration and electron beam dose of 100 kDa hyaluronic acid (HA) and 3 kDa PEG (Y: Hydrogel formation/N: No hydrogel formation).

FIG. 2 is a diagram visually observing whether or not a hydrogel is formed by concentration and electron beam dose of 100 kDa HA and 10 kDa PEG (Y: Hydrogel formation/N: No hydrogel formation).

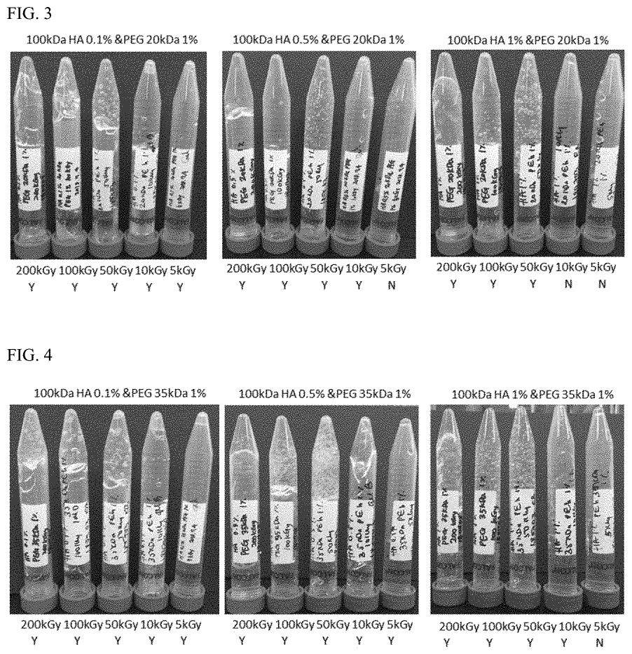

FIG. 3 is a diagram visually observing whether or not a hydrogel is formed by concentration and electron beam dose of 100 kDa HA and 20 kDa PEG (Y: Hydrogel formation/N: No hydrogel formation).

FIG. 4 is a diagram visually observing whether or not a hydrogel is formed by concentration and electron beam dose of 100 kDa HA and 35 kDa PEG (Y: Hydrogel formation/N: No hydrogel formation).

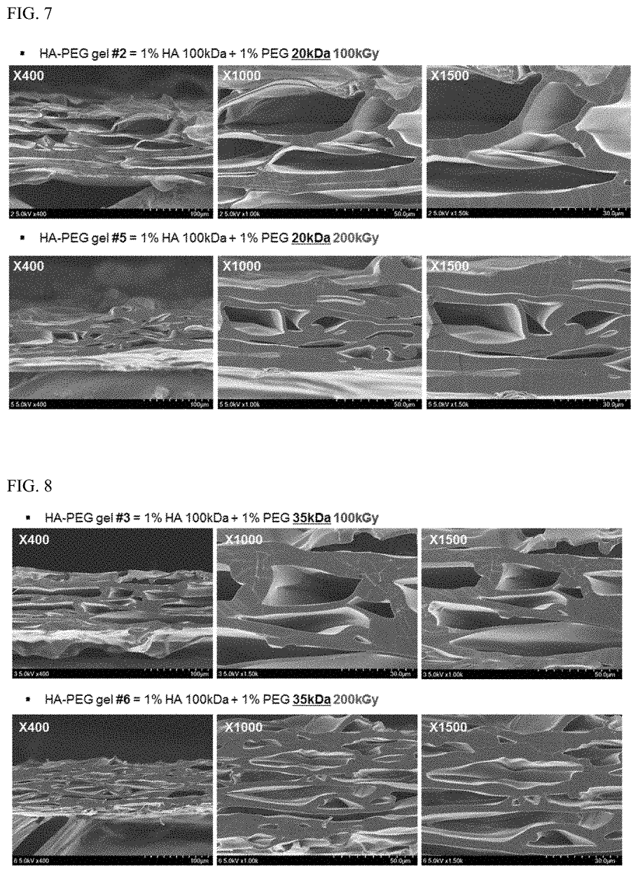

FIG. 5 is a result of confirming whether a hydrogel is formed even when the method of the present invention is applied to mass production.

FIGS. 6 to 9 are diagrams of observing pore sizes of hydrogels formed under indicated conditions with a scanning electron microscope.

FIG. 10 is a result of evaluating a swelling ratio for each solvent of a hydrogel made by irradiating a 100 kGy electron beam to 1% 100 kDa HA and 1% 20 kDa PEG.

FIG. 11 is a diagram illustrating an experiment method for visually observing a degree to be decomposed after left for 1 week by inserting hydrogels formed under indicated conditions into the abdominal cavity of animals.

FIG. 12 is a diagram visually observing a degree to be decomposed after left for 1 week by inserting hydrogels formed under indicated conditions into the abdominal cavity of animals.

FIGS. 13 to 22 are results of visually observing the applicability as a filler in a living body by observing a shape retention degree over time after inserting hydrogels formed under indicated conditions to the forehead or backside of animals.

FIG. 23 is a diagram visually observing an HA-PEG hydrogel sheet formed under indicated conditions to evaluate the efficacy as a wound dressing.

FIG. 24 is a result of visually observing healing degrees of the wound over time after dressing a HA-PEG hydrogel wound dressing according to the present invention on a wound site of a wound animal model.

FIG. 25 is a graph showing a result of measuring areas of the wound over time after dressing HA-PEG hydrogel wound dressings according to the present invention on a wound site of a wound animal model.

FIG. 26 is a result of measuring thicknesses of the healed skin after healing the wound by dressing HA-PEG hydrogel wound dressings according to the present invention on a wound site of a wound animal model.

### MODES FOR THE INVENTION

Hereinafter, the present invention will be described in detail by the following Examples. However, the following Examples are just illustrative of the present invention, and the contents of the present invention are not limited to the following Examples.

### Example 1: Preparation of Hyaluronic Acid (HA)-Polyethylene Glycol (PEG) Hydrogel Through Electron Beam Irradiation

The present inventors conducted experiments under various conditions below to prepare a HA-PEG composite hydrogel only by electron beam irradiation without the addition of a cross-linking agent. Hereinafter, in the example results, a case in which the hydrogel was formed was represented by Y, and a case in which the hydrogel was not formed was represented by N. In addition, the hydrogel was also expressed as a bulk gel.

First, the results according to each concentration and electron beam dose under a condition that 100 kDa of hyaluronic acid and 0.6 kDa to 35 kDa of PEG are mixed may be summarized in Tables and photos below.

In PEG of 0.6 kDa and 1 kDa, it was confirmed that Gel was not formed under all the conditions of performing the experiment.

| 0.6 kDa PEG 1% | | | | | | |
|---|---|---|---|---|---|---|
| 100 kDa HA | 200 kGy | 100 kGy | 50 kGy | 10 kGy | 5 kGy | Note |
| 0.1% | N | N | N | N | N | No gel formation |
| 0.5% | N | N | N | N | N | |
| 1% | N | N | N | N | N | |

| 1 kDa PEG 1% | | | | | | |
|---|---|---|---|---|---|---|
| 100 kDa HA | 200 kGy | 100 kGy | 50 kGy | 10 kGy | 5 kGy | Note |
| 0.1% | N | N | N | N | N | No gel formation |
| 0.5% | N | N | N | N | N | |
| 1% | N | N | N | N | N | |

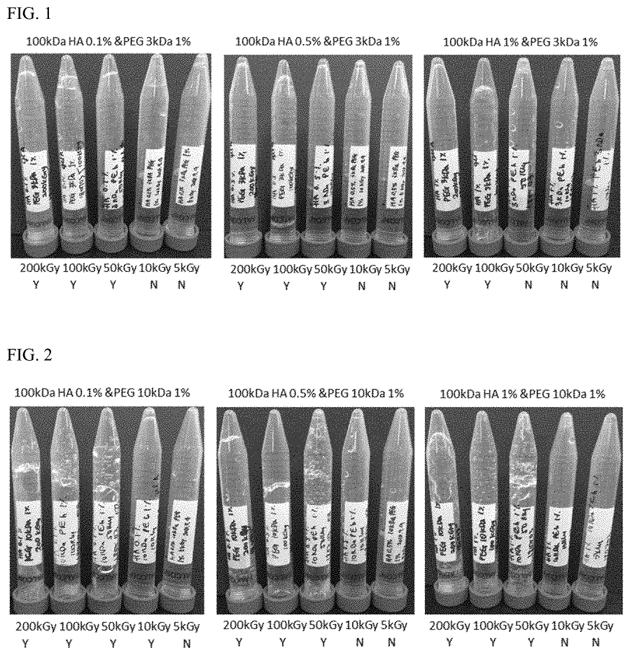

In an experiment using 3 kDa of PEG, the hydrogel started to be formed from the electron beam dose of 50 kGy, and the tendency to form the Gel better as the concentration of hyaluronic acid was lower was confirmed. Although the hydrogel was formed even at 100 kGy, the tendency to form a gel with a slightly higher viscosity than hydrogels formed at 50 and 200 kGy was also confirmed through the photos of the gel corresponding to each condition (FIG. 1). Through this part, it was confirmed that as the electron beam dose increased vaguely, cross-linking increased and thus, a harder Gel was not formed.

| 3 kDa PEG 1% | | | | | | |
|---|---|---|---|---|---|---|
| 100 kDa HA | 200 kGy | 100 kGy | 50 kGy | 10 kGy | 5 kGy | Note |
| 0.1% | Y | Y | Y | N | N | Bulk gel (BG) started to be formed |
| 0.5% | Y | Y | Y | N | N | from electron beam dose of 50 kGy, |
| 1% | Y | Y | N | N | N | and tendency to form Gel better as |
| | | | | | | concentration of hyaluronic acid was |
| | | | | | | lower was confirmed. |
| | | | | | | Bulk gel (BG) was formed in 100 kGy, |
| | | | | | | but tendency to form Gel with higher |
| | | | | | | viscosity than BG formed at 50 and |
| | | | | | | 200 kGy was also confirmed. |

Even in an experiment using 10 kDa of PEG, it was confirmed that the lower the concentration of hyaluronic acid, a hydrogel started to be formed at a lower electron beam dose (10 kGy), and it was confirmed again that the hydrogel was formed at 50 and 100 kGy, but a hydrogel with slightly low hardness was formed at 200 kGy, and thus, even if the cross-linking was vaguely increased as the electron beam dose was increased, a hard hydrogel was not formed.

| 10 kDa PEG 1% | | | | | | |
|---|---|---|---|---|---|---|
| 100 kDa HA | 200 kGy | 100 kGy | 50 kGy | 10 kGy | 5 kGy | Note |
| 0.1% | Y | Y | Y | Y | N | Bulk gel (BG) started to be formed at lower electron |
| 0.5% | Y | Y | Y | N | N | beam dose (10 kGy) as concentration of hyaluronic acid |
| 1% | Y | Y | Y | N | N | was lower. |
| | | | | | | Bulk gel (BG) was formed in 50 and 100 kGy, but Bulk |
| | | | | | | gel (BG) with slightly low hardness was formed at 200 |
| | | | | | | kGy, and thus, it may be confirmed that even if cross- |
| | | | | | | linking was vaguely increased as electron beam dose was |
| | | | | | | increased, a hard hydrogel was not formed. |
| | | | | | | In addition, it is confirmed that the color of a sample |
| | | | | | | formed when the dose increased as concentration of |
| | | | | | | hyaluronic acid increased is gradually yellowish. |

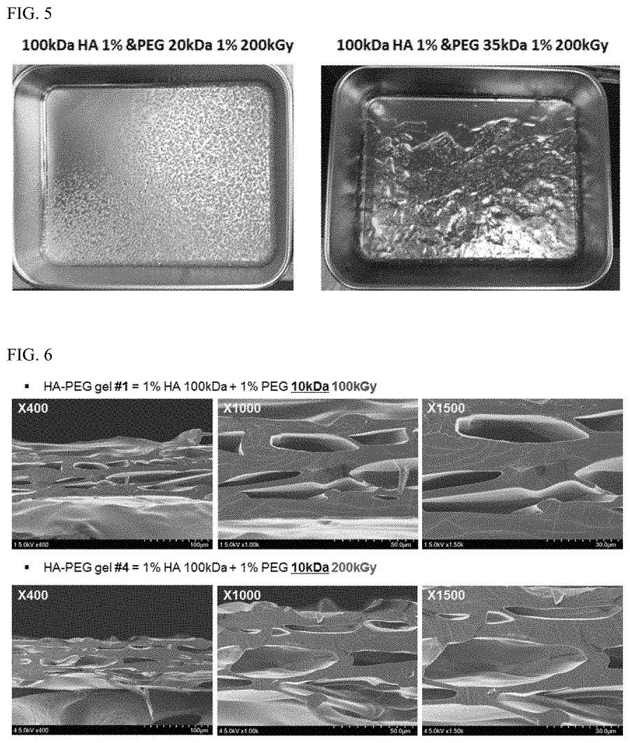

In an experiment using 20 kDa of PEG, when the concentration of hyaluronic acid was 0.1%, the hardness was slightly weak from 5 kGy of a dose, but a hydrogel started to be formed. In addition, at 50 and 100 kGy, a hydrogel was formed, but at 200 kGy, a hydrogel with slightly low hardness was formed, so that the same tendency as the result of the experiment using 10 kDa of PEG was confirmed (FIG. 3).

| 20 kDa PEG 1% | | | | | | |
|---|---|---|---|---|---|---|
| 100 kDa HA | 200 kGy | 100 kGy | 50 kGy | 10 kGy | 5 kGy | Note |
| 0.1% | Y | Y | Y | Y | Y | When the concentration of hyaluronic acid was 0.1%, |
| 0.5% | Y | Y | Y | Y | N | the hardness was slightly weak from 5 kGy of a dose, but |
| 1% | Y | Y | Y | N | N | Bulk gel (BG) started to be formed. |
| | | | | | | At 50 and 100 kGy, Bulk gel (BG) was formed, but at |
| | | | | | | 200 kGy, Bulk gel (BG) with slightly low hardness was |
| | | | | | | formed, so that the same tendency as the result of the |
| | | | | | | experiment using 10 kDa PEG was confirmed. |

Even in an experiment using 35 kDa of PEG, like the result of 20 kDa of PEG, when the concentration of hyaluronic acid was 0.1%, a hydrogel started to be formed from the dose of 5 kGy.

When the concentration of hyaluronic acid was 0.1% and 0.5%, it was confirmed that a hydrogel with strong hardness was formed at 50 and 100 kGy, but a hydrogel with weak hardness was formed at 200 kGy.

| 35 kDa PEG 1% | | | | | | |
|---|---|---|---|---|---|---|
| 100 kDa HA | 200 kGy | 100 kGy | 50 kGy | 10 kGy | 5 kGy | Note |
| 0.1% | Y | Y | Y | Y | Y | Like the result of 20 kDa of PEG, when the |
| 0.5% | Y | Y | Y | Y | Y | concentration of hyaluronic acid was 0.1%, Bulk gel |
| 1% | Y | Y | Y | Y | N | (BG) started to be formed from a dose of 5 kGy. |
| | | | | | | When the concentration of hyaluronic acid was 0.1% |
| | | | | | | and 0.5%, it was confirmed that Bulk gel (BG) was |
| | | | | | | formed at 50 and 100 kGy, but Bulk gel (BG) with |
| | | | | | | slightly weak hardness was formed at 200 kGy. |

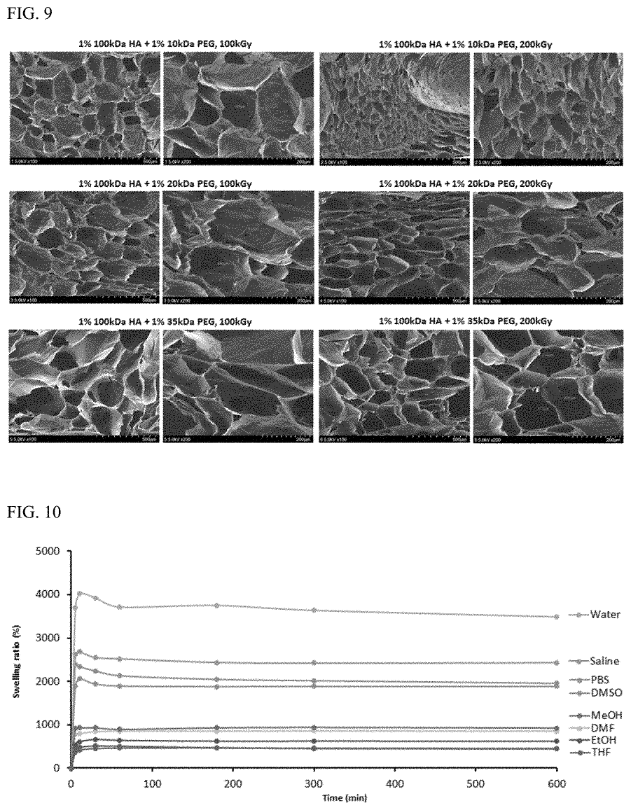

Meanwhile, an experiment for synthesizing a large amount of Gel at once was also conducted by selecting conditions under which some Gels were formed well, and it was confirmed that Gels with the same characteristics may be easily synthesized in a large amount rather than in a small amount (FIG. 5).

Previously, while the concentration was fixed at 1%, the experiment was conducted under a condition in which 100 kDa of hyaluronic acid at 0.1, 0.5, and 1% concentrations was mixed with PEGs of different molecular weights (0.6 to 35 kDa), and on the other hand, while the concentration of 100 kDa hyaluronic acid was fixed at 1%, the concentrations of PEGs having different molecular weights were changed to 0.1%, 0.5%, and 1% to confirm the formation of Gel under each condition.

First, in PEGs of 0.6 kDa and 1 kDa, it was confirmed that Gel was not formed under all the conditions of performing the experiment.

| 100 kDa HA 1% | | | | | | |
|---|---|---|---|---|---|---|
| 0.6 kDa PEG | 200 kGy | 100 kGy | 50 kGy | 10 kGy | 5 kGy | Note |
| 0.1% | N | N | N | N | N | No gel formation |
| 0.5% | N | N | N | N | N | |
| 1% | N | N | N | N | N | |

| 100 kDa HA 1% | | | | | | |
|---|---|---|---|---|---|---|
| 1 kDa PEG | 200 kGy | 100 kGy | 50 kGy | 10 kGy | 5 kGy | Note |
| 0.1% | N | N | N | N | N | No gel formation |
| 0.5% | N | N | N | N | N | |
| 1% | N | N | N | N | N | |

In the experiment using 3 kDa of PEG, Gel was formed only under a condition in which 1% concentration of PEG was mixed together, and it was confirmed that the hydrogel was formed only under the conditions in which 100 kGy and 200 kGy of doses were applied.

| 100 kDa HA 1% | | | | | | |
|---|---|---|---|---|---|---|
| 3 kDa PEG | 200 kGy | 100 kGy | 50 kGy | 10 kGy | 5 kGy | Note |
| 0.1% | N | N | N | N | N | Under a condition in which 1% PEG was mixed |
| 0.5% | N | N | N | N | N | together, it was confirmed that Bulk gel (BG) with |
| 1% | Y | Y | N | N | N | slightly weak hardness was formed under conditions in |
| | | | | | | which 100 kGy and 200 kGy of doses were applied. |

Even in 10 kDa of PEG, it was confirmed that Gel was formed only under the conditions in which 1% concentration of PEG was mixed together, and samples irradiated at 50 and 100 kGy were formed into hydrogels, but a sample irradiated at 200 kGy formed a hydrogel with slightly weak hardness.

| 100 kDa HA 1% | | | | | | |
|---|---|---|---|---|---|---|
| 10 kDa | | | | | | |
| PEG | 200 kGy | 100 kGy | 50 kGy | 10 kGy | 5 kGy | Note |
| 0.1% | N | N | N | N | N | Under a condition in which 1% |
| 0.5% | N | N | N | N | N | PEG was mixed together, it was |
| 1% | Y | Y | Y | N | N | confirmed that samples irradiated |
| | | | | | | with 50 to 100 kGy were formed as |
| | | | | | | Bulk gel (BG), but sample irradiated |
| | | | | | | with 200 kGy was formed as Bulk |
| | | | | | | gel (BG) with slightly weak hardness. |

Even in 20 kDa of PEG, it was confirmed that the samples irradiated at 50 and 100 kGy under the conditions mixed with 1% PEG were formed into hydrogels, but the sample irradiated at 200 kGy was formed into a hydrogel with slightly weak hardness, and the same tendency as the result for the experiment using 10 kDa of PEG was confirmed.

| 100 kDa HA 1% | | | | | | |
|---|---|---|---|---|---|---|
| 20 kDa | | | | | | |
| PEG | 200 kGy | 100 kGy | 50 kGy | 10 kGy | 5 kGy | Note |
| 0.1% | N | N | N | N | N | Like the result for experiment |
| 0.5% | N | N | N | N | N | using 10 kDa PEG, under a condition |
| 1% | Y | Y | Y | N | N | in which 1% PEG was mixed together, |
| | | | | | | it was confirmed that samples irradiated |
| | | | | | | with 50 and 100 kGy were formed as Bulk |
| | | | | | | gel (BG), but sample irradiated with |
| | | | | | | 200 kGy was formed as Bulk gel (BG) with |
| | | | | | | slightly weak hardness. |

It was confirmed that even in 35 kDa of PEG, a hydrogel with slightly weak hardness was formed from the sample irradiated at 10 kGy under the conditions mixed with 1% PEG, and it was confirmed that the samples irradiated at 50 and 100 kGy were formed into hydrogels, but the sample irradiated at 200 kGy was formed into a hydrogel with slightly weak hardness.

To summarize these results, there is a difference depending on the molecular weight of PEG in the Gel formation through electron beam irradiation under the condition mixed with 1% of 100 kDa hyaluronic acid, but it was very important that the concentration of PEG was 1%, even than that. Since it was confirmed that the hardness of the hydrogel was slightly reduced at 200 kGy under the condition mixed with PEG of 10 kDa or more, it was confirmed once again that a harder hydrogel was not formed even if the cross-linking was vaguely increased as the electron beam dose was increased.

| 100 kDa HA 1% | | | | | | |
|---|---|---|---|---|---|---|
| 35 kDa | | | | | | |
| PEG | 200 kGy | 100 kGy | 50 kGy | 10 kGy | 5 kGy | Note |
| 0.1% | N | N | N | N | N | Under a condition in which 1% |
| 0.5% | N | N | N | N | N | PEG was mixed together, it was |
| 1% | Y | Y | Y | Y | N | confirmed that even sample irradiated |
| | | | | | | with 10 kGy, Bulk gel (BG) with |
| | | | | | | slightly weak hardness was formed. |
| | | | | | | It was confirmed that samples irradiated |
| | | | | | | with 50 and 100 kGy were formed as |
| | | | | | | Bulk gel (BG), but sample irradiated |
| | | | | | | with 200 kGy was similarly formed as |
| | | | | | | Bulk gel (BG) with slightly weak hardness. |

Next, an experiment was conducted to compare electron beam irradiation energies. So far, the experiment was conducted by fixing all the electron beam irradiation energies to 1 MeV, but an experiment was conducted to confirm which difference in Gel formation was exhibited under the electron beam irradiation condition, in which the energy was changed to 2.5 MeV and other conditions were kept the same.

First, in 0.6 kDa PEG, it was confirmed that Gel was not formed under all the conditions of performing the experiment.

| 100 kDa HA 1% | | | | | | | | | |
|---|---|---|---|---|---|---|---|---|---|
| 0.6 kDa | Beam | | | | | | | | |
| PEG | energy | | 300 kGy | 200 kGy | 100 kGy | 50 kGy | 10 kGy | 5 kGy | Note |
| 0.5% | 1 | MeV | N | N | N | N | N | N | No gel |
| | 2.5 | MeV | N | N | N | N | N | N | formation |
| 1% | 1 | MeV | N | N | N | N | N | N | |
| | 2.5 | MeV | N | N | N | N | N | N | |

Next, in an experiment using 1 kDa of PEG, a difference according to the electron beam irradiation energy intensity was confirmed, but it was confirmed that the hydrogel was formed when 300 kGy of the electron beam was irradiated to 1% concentration of 1 kDa PEG at 2.5 MeV, unlike at 1 MeV.

| 100 kDa HA 1% | | | | | | | | | |
|---|---|---|---|---|---|---|---|---|---|
| 1 kDa | Beam | | | | | | | | |
| PEG | energy | | 300 kGy | 200 kGy | 100 kGy | 50 kGy | 10 kGy | 5 kGy | Note |
| 0.5% | 1 | MeV | N | N | N | N | N | N | Unlike at 1 MeV, at 2.5 MeV, Bulk |
| | 2.5 | MeV | N | N | N | N | N | N | gel (BG) was formed when 300 kGy |
| 1% | 1 | MeV | N | N | N | N | N | N | of electron beam was irradiated to |
| | 2.5 | MeV | Y | N | N | N | N | N | 1% PEG. |

In the experiment using 3 kDa of PEG, it was confirmed that Gel was not formed in all conditions using 0.5% PEG, and in the condition using 1% PEG, there was the same tendency that the hydrogels were formed from 100 kGy at both 1 MeV and 2.5 MeV.

| 100 kDa HA 1% | | | | | | | | | |
|---|---|---|---|---|---|---|---|---|---|
| 3 kDa | Beam | | | | | | | | |
| PEG | energy | | 300 kGy | 200 kGy | 100 kGy | 50 kGy | 10 kGy | 5 kGy | Note |
| 0.5% | 1 | MeV | N | N | N | N | N | N | Gel was not formed in all |
| | 2.5 | MeV | N | N | N | N | N | N | conditions of 0.5% PEG. |
| 1% | 1 | MeV | Y | Y | Y | N | N | N | There was the same tendency that |
| | 2.5 | MeV | Y | Y | Y | N | N | N | Bulk gel (BG) was formed from 100 |
| | | | | | | | | | kGy at both 1 MeV and 2.5 MeV. |

Even in the experiment using 10 kDa of PEG, it was confirmed that Gel was not formed in all conditions using 0.5% PEG, and in the condition using 1% PEG, there was the same tendency that the hydrogels were formed from 50 kGy at both 1 MeV and 2.5 MeV.

| 100 kDa HA 1% | | | | | | | | | |
|---|---|---|---|---|---|---|---|---|---|
| 10 kDa | Beam | | | | | | | | |
| PEG | energy | | 300 kGy | 200 kGy | 100 kGy | 50 kGy | 10 kGy | 5 kGy | Note |
| 0.5% | 1 | MeV | N | N | N | N | N | N | Gel was not formed in all |
| | 2.5 | MeV | N | N | N | N | N | N | conditions of 0.5% PEG. |
| 1% | 1 | MeV | Y | Y | Y | Y | N | N | There was the same tendency that |
| | 2.5 | MeV | Y | Y | Y | Y | N | N | Bulk gel (BG) was formed from 50 |
| | | | | | | | | | kGy at both 1 MeV and 2.5 MeV. |

Similarly, even in the experiment using 20 kDa PEG, Gel was not formed under all conditions using 0.5% PEG, but in the condition using 1% PEG, a difference was confirmed according to the intensity of the electron beam irradiation energy, and unlike 1 MeV, a difference in hydrogel formation from 10 kGy was confirmed at 2.5 MeV.

| 100 kDa HA 1% | | | | | | | | | |
|---|---|---|---|---|---|---|---|---|---|
| 20 kDa | Beam | | | | | | | | |
| PEG | energy | | 300 kGy | 200 kGy | 100 kGy | 50 kGy | 10 kGy | 5 kGy | Note |
| 0.5% | 1 | MeV | N | N | N | N | N | N | Gel was not formed in all |
| | 2.5 | MeV | N | N | N | N | N | N | conditions of 0.5% PEG. |
| | 1 | MeV | Y | Y | Y | Y | N | N | It was confirmed that there was a |
| 1% | 2.5 | MeV | Y | Y | Y | Y | Y | N | difference in that Bulk gel (BG) |
| | | | | | | | | | was formed from 10 kGy at 2.5 MeV. |

Even in the experiment using 35 kDa of PEG, similarly, it was confirmed that Gel was not formed in all conditions using 0.5% PEG, and in the condition using 1% PEG, the hydrogels were formed from 10 kGy at both 1 MeV and 2.5 MeV. However, it was confirmed that the hydrogels formed at 200 and 300 kGy using 2.5 MeV had somewhat more hardness than the hydrogel formed at 1 MeV.

| 100 kDa HA 1% | | | | | | | | | |
|---|---|---|---|---|---|---|---|---|---|
| 35 kDa | Beam | | | | | | | | |
| PEG | energy | | 300 kGy | 200 kGy | 100 kGy | 50 kGy | 10 kGy | 5 kGy | Note |
| 0.5% | 1 | MeV | N | N | N | N | N | N | Gel was not formed in all conditions of 0.5% PEG. |
| | 2.5 | MeV | N | N | N | N | N | N | Bulk gel (BG) was formed from 10 |
| 1% | 1 | MeV | Y | Y | Y | Y | Y | N | kGy at both 1 MeV and 2.5 MeV, but it |
| | 2.5 | MeV | Y | Y | Y | Y | Y | N | was confirmed that Bulk gel (BG) |
| | | | | | | | | | formed at 200 and 300 kGy at 2.5 MeV |
| | | | | | | | | | had somewhat more hardness than Bulk |
| | | | | | | | | | gel (BG) formed at 1 MeV. |

Summarizing the above results, it could be predicted that as the intensity of the electron beam irradiation energy increased, the energy was transmitted more deeply into the sample, so that the Gel was formed easier.

Next, using 1% of 2500 kDa hyaluronic acid having a greater molecular weight, an electron beam irradiation experiment was conducted in the range of irradiation dose of 5 to 200 kGy under conditions mixed with PEGs of different concentrations.

In PEG of 0.6 kDa and 1 kDa, it was confirmed that Gel was not formed under all the conditions of performing the experiment.

| 1% 2500 kDa HA | | | | | | |
|---|---|---|---|---|---|---|
| 0.6 kDa | | | | | | |
| PEG | 200 kGy | 100 kGy | 50 kGy | 10 kGy | 5 kGy | Note |
| 0.1% | N | N | N | N | N | Gel was not formed |
| 0.5% | N | N | N | N | N | under all the conditions |
| 1% | N | N | N | N | N | (NG). |

| 1% 2500 kDa HA | | | | | | |
|---|---|---|---|---|---|---|
| 1 kDa | | | | | | |
| PEG | 200 kGy | 100 kGy | 50 kGy | 10 kGy | 5 kGy | Note |
| 0.1% | N | N | N | N | N | Gel was not formed |
| 0.5% | N | N | N | N | N | under all the conditions |
| 1% | N | N | N | N | N | (NG). |

In an experiment using 3 kDa PEG, it was confirmed that a hydrogel was formed under the conditions of mixing 1% PEG and irradiating 100 kGy, whereas it was confirmed that when 200 kGy was irradiated, a slightly less hard hydrogel was formed. It was confirmed that even if the cross-linking was further increased as the electron beam dose was increased, a hard hydrogel was not formed.

| 1% 2500 kDa HA | | | | | | |
|---|---|---|---|---|---|---|
| 3 kDa | | | | | | |
| PEG | 200 kGy | 100 kGy | 50 kGy | 10 kGy | 5 kGy | Note |
| 0.1% | N | N | N | N | N | Bulk gel (BG) was formed under |
| 0.5% | N | N | N | N | N | conditions of mixing 1% 3 kDa |
| 1% | Y | Y | N | N | N | PEG and irradiating 100 kGy. |
| | | | | | | When 200 kGy was irradiated, |
| | | | | | | the hardness of BG was slightly weak. |

Even in 10 kDa of PEG, the Gel was formed only in the condition in which 1% PEG was mixed, but unlike this, it was confirmed that the hydrogel was formed from a 50 kGy irradiated condition, and it was confirmed that the hydrogels were formed at both 100 kGy and 200 kGy.

| 1% 2500 kDa HA | | | | | | |
|---|---|---|---|---|---|---|
| 10 kDa | | | | | | |
| PEG | 200 kGy | 100 kGy | 50 kGy | 10 kGy | 5 kGy | Note |
| 0.1% | N | N | N | N | N | Bulk gel (BG) was formed |
| 0.5% | N | N | N | N | N | from conditions of mixing |
| 1% | Y | Y | Y | N | N | 1% 10 kDa PEG and irradiating 50 kGy. |

It was confirmed that even in 20 kDa PEG, the hydrogel was formed from a condition in which 1% of 20 kDa PEG was mixed and 50 kGy was irradiated in the same tendency as in the condition in which 10 kDa PEG was mixed, and it was confirmed that the hydrogels were formed at both 100 kGy and 200 kGy. It was confirmed that 10 kDa and 20 kDa of PEGs did not show a significant difference under the electron beam irradiation experiment conditions.

| 1% 2500 kDa HA | | | | | | |
|---|---|---|---|---|---|---|
| 20 kDa | | | | | | |
| PEG | 200 kGy | 100 kGy | 50 kGy | 10 kGy | 5 kGy | Note |
| 0.1% | N | N | N | N | N | In the same as result using |
| 0.5% | N | N | N | N | N | 10 kDa PEG, Bulk gel (BG) |
| 1% | Y | Y | Y | N | N | was formed from conditions |
| | | | | | | of mixing 1% PEG and irradiating |
| | | | | | | 50 kGy. |

Even in 35 kDa PEG, the result was similar to those of 10 kDa and 20 kDa of PEGs, but hydrogels started to be formed from a condition in which 1% of PEG was mixed and 10 kGy was irradiated. Therefore, it was confirmed that as the molecular weight of the mixed PEG was increased, the Gel started to be formed from a lower electron beam dose.

| 1% 2500 kDa HA | | | | | | |
|---|---|---|---|---|---|---|
| 35 kDa | | | | | | |
| PEG | 200 kGy | 100 kGy | 50 kGy | 10 kGy | 5 kGy | Note |
| 0.1% | N | N | N | N | N | The result was similar to the |
| 0.5% | N | N | N | N | N | result in 10 kDa and 20 kDa PEGs, |
| 1% | Y | Y | Y | Y | N | but Bulk gel (BG) started to be |
| | | | | | | formed from conditions of mixing |
| | | | | | | 1% PEG and irradiating 10 kGy. |

When summarizing the result, it was confirmed that the tendency of the result using 100 kDa hyaluronic acid are almost the same as a whole, and it was confirmed that even if 2500 kDa hyaluronic acid much greater than a molecular weight was used, Gel was formed only under a condition in which 1% of PEG was mixed. In addition, it was confirmed that as the molecular weight of the mixed PEG was increased, the Gel also started to be formed from a lower electron beam dose. In addition, in an experiment using 3 kDa of PEG, it was confirmed that a hydrogel was formed under a 100 kGy irradiated condition, but when 200 kGy was irradiated, a slightly less hard hydrogel was formed, so that in the same manner as the above experiments, it was confirmed once again that even if the cross-linking was increased as the electron beam dose was increased, a harder hydrogel was not formed.

### Example 2: Observation of Pores and Confirmation of Water Retention of HA-PEG Hydrogel Formed Through Electron Beam Irradiation

In order to confirm the pore size of a hyaluronic acid-PEG hydrogel formed through electron beam irradiation, a hydrogel sample was freeze-dried, cut in half using a blade, and then coated with Osmium, and thereafter, the size and thickness of the hole were confirmed through a scanning electron microscope (SEM). In an analysis experiment through the SEM, a hyaluronic acid-PEG hydrogel prepared by irradiating 100 kGy and 200 kGy to 1% 10 kDa PEG, 20 kDa PEG, and 35 kDa PEG along with 1% 100 kDa hyaluronic acid was used.

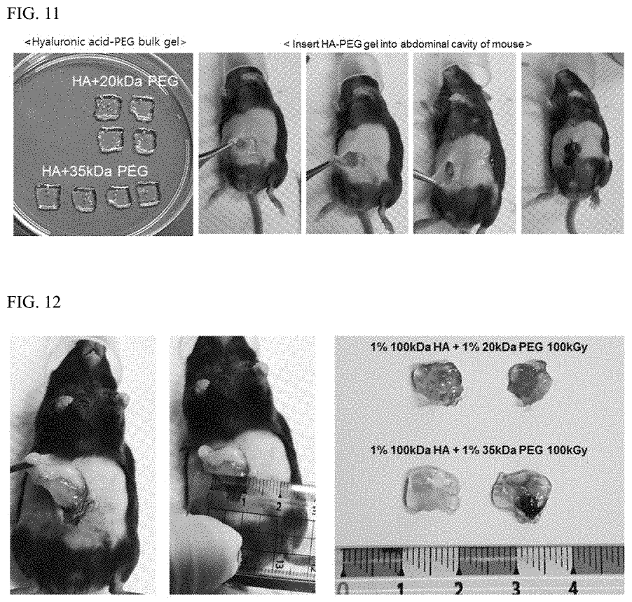

First, when describing the hyaluronic acid-PEG hydrogel prepared by irradiating 100 kGy and 200 kGy to 1% 10 kDa PEG mixed with 1% 100 kDa hyaluronic acid through the SEM, it was confirmed that the thickness of the sample prepared through freeze-drying was slightly thinner as the electron beam dose was increased, and it was confirmed that there was no significantly difference in size of the pore or thickness between films forming pores (FIG. 6).

Next, when describing the hyaluronic acid-PEG hydrogel prepared by irradiating 100 kGy and 200 kGy to 1% 20 kDa PEG mixed with 1% 100 kDa hyaluronic acid, it was confirmed once again that the thickness of the sample formed through freeze-drying was thinner as the electron beam dose was increased. It was confirmed that the size of the pore also decreased somewhat and the thickness of the films forming the pores became somewhat thicker (FIG. 7).

Finally, the hyaluronic acid-PEG hydrogel prepared by irradiating 100 kGy and 200 kGy to 1% 35 kDa PEG mixed with 1% 100 kDa hyaluronic acid was confirmed, and like the result for 20 kDa PEG, it was confirmed that the thickness of the freeze-dried sample was thinner as the electron beam dose was increased, and it was confirmed that the size of the pore also decreased and the thickness of the films forming the pores had no significant difference (FIG. 8).

Next, the amount of the sample to be freeze-dried was sufficiently used to make the thickness sufficiently thick, and then an experiment was conducted to confirm a difference according to the molecular weight and electron beam dose of PEG mixed with hyaluronic acid through a scanning electron microscope through the same method for the same sample. As a result, it was confirmed that the size of the pores increased as the molecular weight of the mixed PEG increased, and it was also clearly confirmed the tendency that the size of the pores decreased as the electron beam dose increased (FIG. 9).

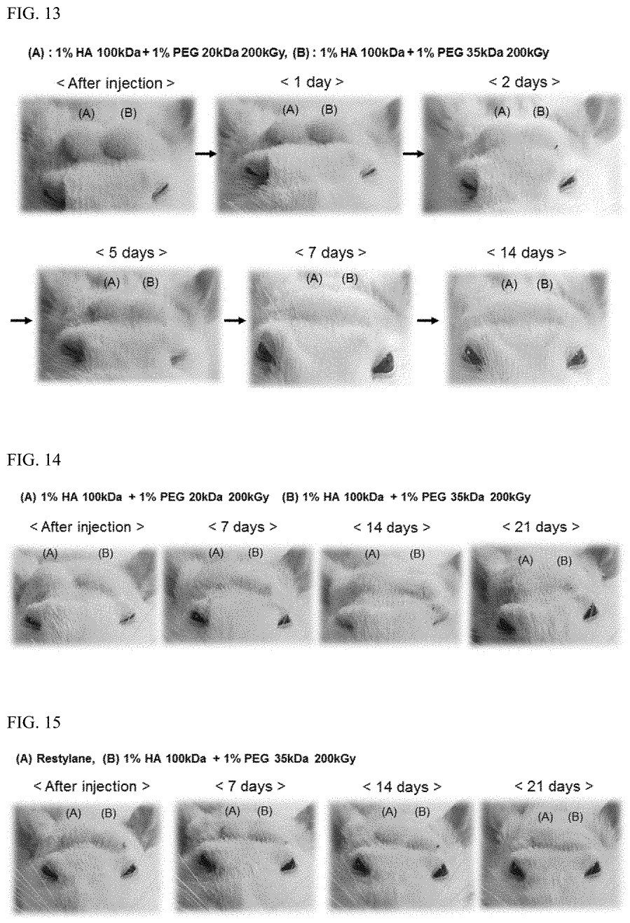

Next, an experiment was conducted to confirm how much the hyaluronic acid-PEG hydrogel may keep a solvent (swelling), and the hydrogel formed by irradiating an electron beam of 100 kGy to 1% 100 kDa hyaluronic acid and 1% 20 kDa PEG was freeze-dried to match the same size, and then added in various types of solvents such as water, saline, PBS, DMSO, MeOH, DMF, EtOH, and THF to monitor how much each solvent was kept over time and the size and weight thereof to 10 hours. As a result, the weight of the hyaluronic acid-PEG hydrogel reached a maximum value within 5 minutes to quickly absorb most solvents, but the difference thereof occurred according to a type of solvent, and it was confirmed that the highest swelling was made in water other than in other solvents, and next, the swelling ability of the hyaluronic acid-PEG hydrogel was higher in water-base solvents in order of saline and PBS. Such a result is predicted as a result derived from the excellent water retention ability of hyaluronic acid included in the hyaluronic acid-PEG hydrogel (FIG. 10).

### Example 3: Confirmation of In Vivo Decomposition of HA-PEG Hydrogel

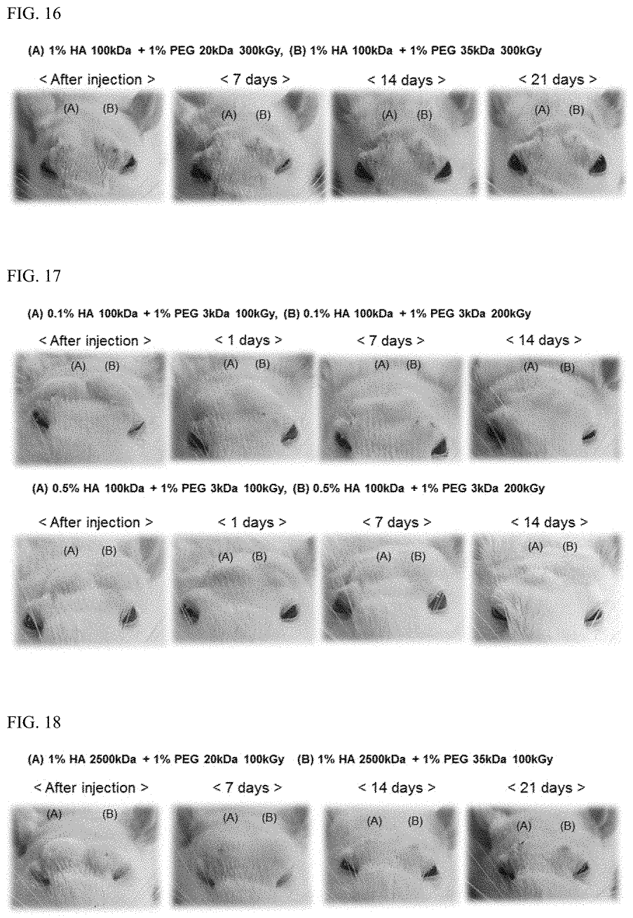

Next, an experiment was conducted to confirm the degree of decomposing the hyaluronic acid-PEG hydrogel in the body, and two hydrogels formed by irradiating an electron beam of 100 kGy to 1% 100 kDa hyaluronic acid and 1% 20 kDa PEG and 1% 100 kDa hyaluronic acid and 1% 35 kDa PEG were cut to a width and a length of 1 cm, respectively, and then inserted into the abdominal cavity of C57BL/6J mice and the abdomen was cut and open after 1 week, and the decomposed degree was confirmed (FIG. 11).

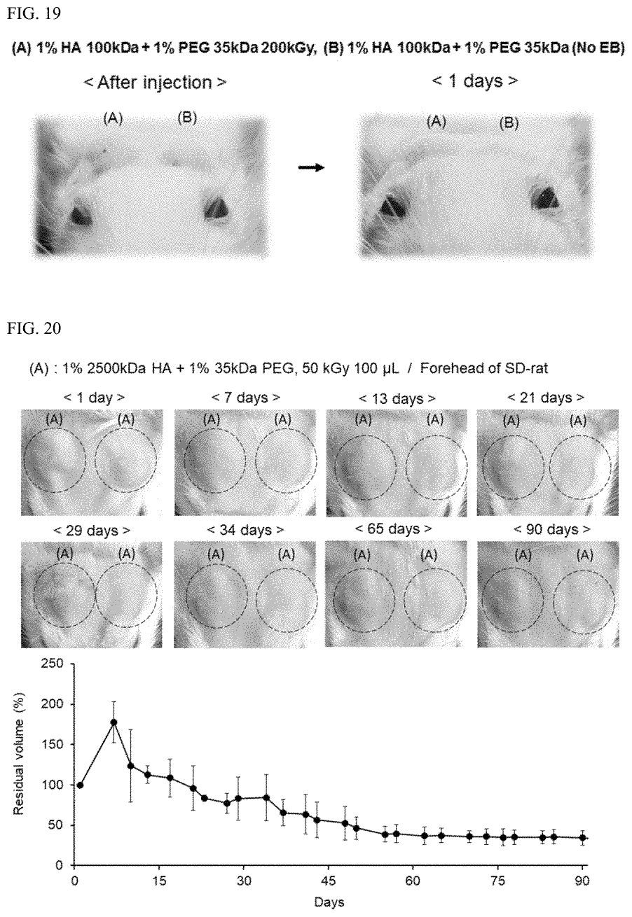

As a result, it was confirmed that the decomposed degree was somewhat different depending on the molecular weight of the PEG mixed with hyaluronic acid. After 1 week, when the inserted hyaluronic acid-PEG hydrogel was taken out and checked, it was confirmed that the hyaluronic acid-PEG hydrogel mixed with PEG of 20 kDa, which had a slightly smaller molecular weight than the hyaluronic acid-PEG hydrogel mixed with PEG of 35 kDa, was decomposed somewhat faster and the size thereof was reduced. Through these results, it could be predicted that the smaller the molecular weight of the PEG mixed in the hyaluronic acid-PEG hydrogel, the faster the tendency to be decomposed in the body (FIG. 12).

### Example 4: Confirmation of Applicability as Filler in Living Body of HA-PEG Hydrogel

In order to perform the efficacy evaluation for using the hyaluronic acid-PEG hydrogel synthesized through electron beam irradiation as a filler, an animal model was prepared using SD-rat. Under gas anesthesia, the hair on the forehead of the SD-rat was removed cleanly, and sample types formed by irradiating an electron beam to the left and right sides were different from each other and injected using a 29 G syringe by 50 μL, and then the effects between the two samples were compared with each other through photos.

First, hydrogels formed by irradiating 200 kGy of an electron beam to 1% 100 kDa hyaluronic acid and 1% 20 kDa PEG and 1% 100 kDa hyaluronic acid and 1% 35 kDa PEG were injected to the left and right sides of the forehead of SD-rat, respectively.

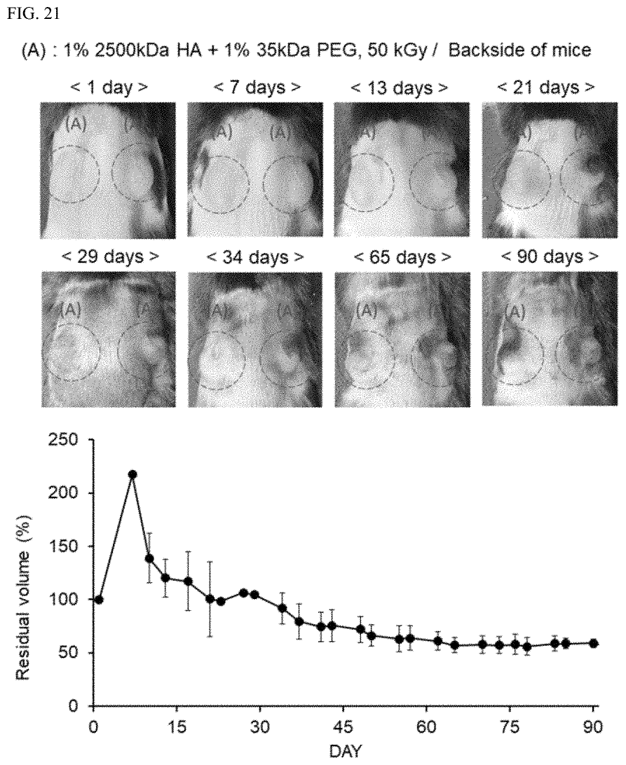

The result of maintaining the volume while both samples remained until 14 days was confirmed, but there was no significant difference between the two samples. Therefore, it was confirmed that the efficacy as a filler of the hydrogel formed in the molecular weight range of 20 kDa and 35 kDa of PEG mixed with hyaluronic acid was similar (FIG. 13).

Thereafter, the same sample was injected into another SD-rat forehead, and the result thereof was confirmed once more, and monitoring was conducted for 3 weeks. As a result, as in the previous experiment, the volume was clearly maintained in both samples until 14 days, but at 3 weeks, it was confirmed that the volume decreased significantly compared to the first injection and somewhat clear observation was difficult by photos, but the volume remained somewhat in the two samples when touched by the hand. Even in repeated experiments, the efficacy of the hyaluronic acid-PEG hydrogel as the filler may be confirmed once again (FIG. 14).

Next, an experiment was conducted by comparing a product called Skinboosters of Restylane Co., Ltd., which has been actually used as a filler in clinical practice, with hyaluronic acid-PEG hydrogel formed by irradiating a 200 kGy electron beam to 1% 100 kDa hyaluronic acid and 1% 35 kDa PEG. As a result, it was confirmed that the degree of maintaining the volumes at both samples was gradually decreased over time, and when monitoring was performed for up to 3 weeks, the volumes of the both samples were maintained. Although the efficacy and durability of the filler was slightly different depending on the type and purpose of filler products which have been used in the related art, it was confirmed that there was a filler effect similar to that of the Skinboosters product of Restylane Co., Ltd. used in the experiment (FIG. 15).

Next, the efficacy as a filler was confirmed by using a hyaluronic acid-PEG hydrogel formed under a condition that the electron beam dose was increased to 300 kGy instead of 200 kGy, and as a result, it was confirmed that the volume was slowly reduced compared with first injection, but when the monitoring was performed up to 3 weeks, the volumes of the two samples were maintained. However, since the elasticity and hardness of the hyaluronic acid-PEG hydrogel formed according to an electron beam dose are somewhat different, the sample using 300 kGy was not soft enough to be used as a filler compared to the sample using 200 kGy. Due to this, it was confirmed that the injection was somewhat stiff, and the shape of the injected filler was slightly distorted rather than a smooth round shape (FIG. 16).

A filler efficacy experiment was also conducted even using the hyaluronic acid-PEG hydrogel prepared when the concentration of hyaluronic acid and the molecular weight of PEG used in the electron beam irradiation experiment were varied. Hydrogels prepared by irradiating 100 kGy and 200 kGy of electron beams to 0.1% or 0.5% 100 kDa hyaluronic acid and 1% 3 kDa PEG were injected into the foreheads of two SD-rats, respectively, but it was confirmed that since the efficacy as a filler was insufficient, the volume was rapidly reduced after injection and almost no volume remained after 14 days. Therefore, it was confirmed that an appropriate concentration of hyaluronic acid used in the electron beam irradiation experiment was required (FIG. 17).

In addition, even when the experiment was conducted by changing the molecular weight of hyaluronic acid used in the electron beam irradiation experiment to 2500 kDa, a hyaluronic acid-PEG hydrogel was synthesized, and when a filler efficacy experiment was conducted using the synthesized hyaluronic acid-PEG hydrogel, it was confirmed that all the volumes of the samples were maintained until 3 weeks, and the results were almost similar to the results in the hyaluronic acid-PEG hydrogel prepared using 100 kDa hyaluronic acid (FIG. 18).

Of course, in the hyaluronic acid-PEG hydrogel synthesis using 2500 kDa hyaluronic acid, since 100 kGy was used as the electron beam dose, it was difficult to be clearly compared with a 100 kDa hyaluronic acid-based hyaluronic acid-PEG hydrogel using 200 kGy, but it was confirmed that the 2500 kDa-based hyaluronic acid-PEG hydrogel, which was expected that an efficacy as the filler was very excellent due to a large molecular weight, was also sufficiently used as a filler.

In addition, in the case of a hyaluronic acid+PEG sample that was not irradiated with electron beams, it was confirmed that the volume quickly disappeared even after one day after injection into the SD-rat forehead, and as a result, it was confirmed that the electron beam irradiation process was required for hydrogel formation through cross-linking between hyaluronic acid and PEG (FIG. 19).

Next, the efficacy as a filler of the hydrogel prepared with an electron beam dose of 50 kGy was evaluated.

Under gas anesthesia, the hair on the forehead of SD-rat was clearly removed and a bulk gel prepared by irradiating a 50 kGy electron beam to 1% 2500 kDa hyaluronic acid and 1% 35 kDa PEG was injected to the left and right sides of the forehead of SD-rat by 100 μL using a 29 G syringe. Thereafter, as a result of observing the volumes of both sides injected with the filler until total 90 days using photos and a caliper, it was confirmed that the volume was increased to about 1.8 times after the first injection, and then the volume was gradually decreased, but the volume thereof was maintained at about 35% of the volume at the time of the first injection even after 90 days (FIG. 20).

In addition to SD-rat, an experiment using C57BL assay mice was additionally conducted. After the hair on the backside of the mouse was clearly removed, the prepared bulk gel sample was injected into the left and right sides of the backside of the mouse by 100 μL using a 29 G syringe, respectively. Similarly, as a result of monitoring for 90 days, it was confirmed that the volume was maintained at about 59% of the volume at the time of the first injection even after 90 days (FIG. 21).

In addition, an experiment was conducted by comparing the efficacy as a filler with a Skinboosters product of Restylane Co., Ltd., which has been actually used as a filler in clinical practice. After the hair on the backside of the BALB/c mouse was clearly removed, the Restylane's filler product was injected to the left side of the hair-removed backside, and a bulk gel prepared by irradiating 50 kGy electron beam to 1% 2500 kDa hyaluronic acid and 1% 35 kDa PEG was injected to the right side thereof by 100 μL. As a result of monitoring for a total of 60 days, it was confirmed that the volumes of both samples were gradually decreased over time, and it was confirmed through photos that the volumes of both samples were maintained until 60 days. As a result of observation, it was confirmed that the bulk gel prepared according to the method of the present invention exhibited better filler efficacy than the Restylane's product (FIG. 22).

### Example 5: Efficacy of HA-PEG Hydrogel Wound Dressing

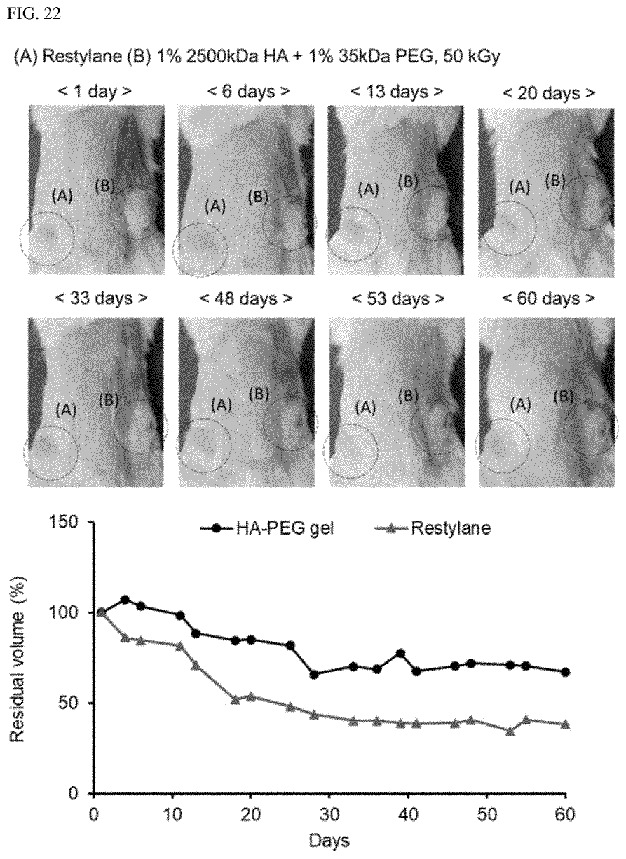

In order to evaluate the efficacy of the HA-PEG hydrogel according to the present invention as a wound dressing, a wound experiment model was first prepared. After wounds were made on both the left and right sides of the backside of BALB/c nude mice using a biopsy punch with a diameter of 8 mm, 4 types of HA-PEG hydrogel samples were placed on the wounds and dressed with a tape, and then the hydrogel samples were replaced every 3 days to monitor the size of the wound for 13 days (FIG. 23). In a wound dressing efficacy comparison experiment, the experiment was performed by using a non-treated control group as a control group.

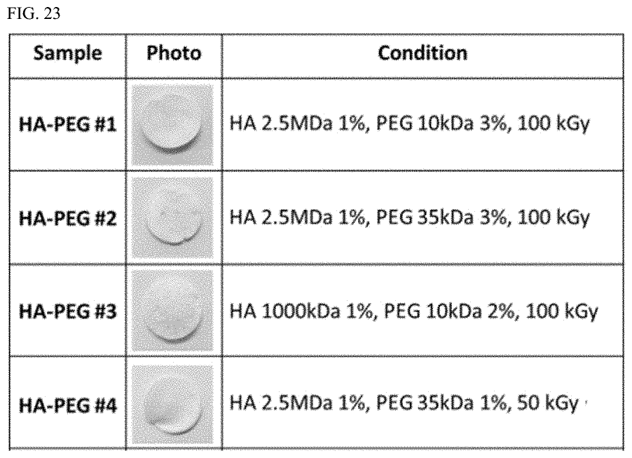

When the hydrogel samples were replaced every 3 days, the sizes of the wounds were checked, and monitoring was performed for a total of 13 days, the degree of wound healing and skin regeneration was observed in each experimental group. As a result, in group treated with HA-PEG #3 and HA-PEG #4 hydrogels, it was confirmed that the wound healing rate was the fastest, and the size of the scar was small on 13 day (FIG. 24).

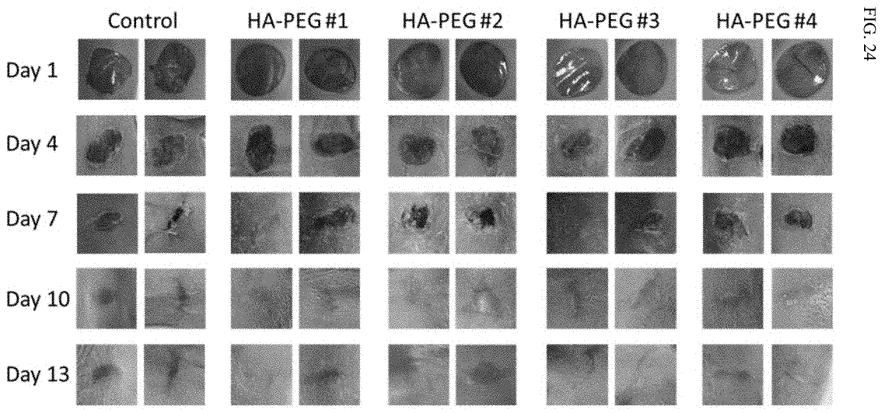

When the areas of the wounds of each group by date during the monitoring period were summarized in a graph, as can be seen in FIG. 25, in the group treated with the hydrogels of HA-PEG #3 and HA-PEG #4, compared to the Control group, on 13 day, it was confirmed that the area of the wound and the size of the scar remained smaller 1.65 times and 2.3 times, respectively.

As such a result, it was determined that HA promoted the formation of a structural skeleton through interaction with fibrin and thrombus in an initial inflammatory reaction caused by wounds, facilitated the movement of cells required for wound healing and formed a network in the granulation tissue at the same time to induce cell proliferation and organization of cells, and helped keratinocytes, core cells of the epidermis, to grow well to make these results.

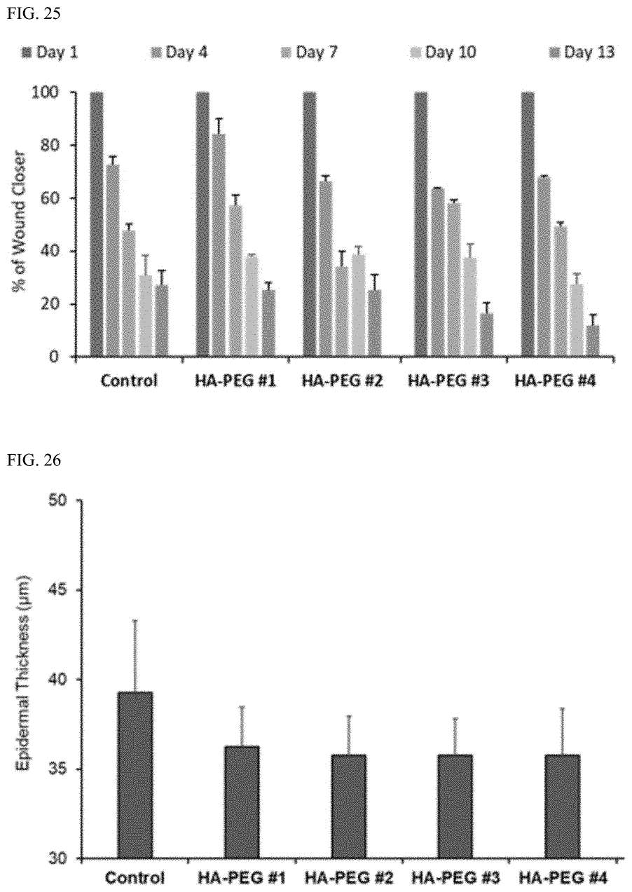

After measuring the wound areas, the skin tissue was extracted and the thickness of the skin tissue was measured through H&E staining, and as a result, in a group treated with all HA-PEG hydrogels, it was confirmed that a thinner skin thickness was confirmed than in the Control group. Considering the characteristics that the thickness and shape of the wound due to the deposition of collagen were non-uniform as the thickness of the skin was increased, it was confirmed that the HA-PEG hydrogels prevented the deposition of collagen in the skin to exhibit the wound dressing effect (FIG. 26).

### INDUSTRIAL APPLICABILITY

Since the hydrogel of the present invention is prepared by inducing inter-molecular and/or intra-molecular cross-linking of hyaluronic acid and polyethylene glycol through an electron beam, there is no risk of toxicity in the human body due to the mixing of an organic solvent or a cross-linking agent, a separate purification process is not required during the preparing process, and mass production is possible only by irradiating the electron beam in a short time, so that it is very excellent even in terms of productivity. In addition, since the hydrogel of the present invention has very excellent biocompatibility, it may be very usefully used in the development of cell carriers, drug carriers, anti-adhesion agents, cell supports, dental fillers, orthopedic fillers, wound dressings, skin fillers, or the like, and thus industrial applicability is very high.

What is claimed is:

1. A hydrogel formed solely by inter-molecular cross-linking, intra-molecular cross-linking, or inter-molecular and intra-molecular cross-linking of hyaluronic acid and polyethylene glycol (PEG).

2. The hydrogel of claim 1, wherein the inter-molecular cross-linking and the intra-molecular cross-linking are formed by radiation irradiation.

3. The hydrogel of claim 2, wherein the radiation is at least one selected from the group consisting of gamma rays, ultraviolet rays, X-rays and electron beams.

4. The hydrogel of claim 1, wherein the hydrogel is prepared by a method comprising the following steps:
(a) preparing a solution by adding hyaluronic acid and polyethylene glycol to water; and
(b) inducing the cross-linking of said materials by irradiating radiation to the solution prepared in step (a).

5. The hydrogel of claim 4, wherein the polyethylene glycol has a molecular weight of 2 to 50 kDa and is added to water at a concentration of 0.6 to 3% (w/v).

6. The hydrogel of claim 4, wherein the hyaluronic acid has a molecular weight of 50 to 3000 kDa and is added to water at a concentration of 0.05 to 3% (w/v).

7. The hydrogel of claim 4, wherein the radiation dose is 2 to 500 kGy.

8. The hydrogel of claim 4, wherein the energy intensity of the radiation is 0.5 to 20 MeV.

9. A method for preparing a hydrogel formed solely by inter-molecular cross-linking, intra-molecular cross-linking, or inter-molecular and intra-molecular cross-linking of hyaluronic acid and polyethylene glycol (PEG), comprising the following steps:
(a) preparing a solution by adding hyaluronic acid and polyethylene glycol to water; and
(b) inducing the cross-linking of said materials by irradiating radiation to the solution prepared in step (a).

10. A cell carrier, a drug carrier, an anti-adhesion agent, a cell support, a dental filler, an orthopedic filler, a wound dressing or a skin filler comprising the hydrogel of any one of claims 1 to 8.

11. A sheet-type, cream-type, gel-type, or spray-type wound dressing comprising the hydrogel of any one of claims 1 to 8.

12. A composition for skin application of a wound site comprising the hydrogel of any one of claims 1 to 8 as an active ingredient.

13. Use of the hydrogel of any one of claims 1 to 8 for preparing an agent for skin application of a wound site.

14. A method for treating a wound site by applying an effective amount of a composition comprising the hydrogel of any one of claims 1 to 8 as an active ingredient onto the skin of a subject in need thereof.
